(19) United States
(12) Patent Application Publication
(10) Pub. No.: US 2017/0340868 A1
(43) Pub. Date: Nov. 30, 2017

(54) PROSTATE DILATION CATHETER

(71) Applicant: BEIJING UNIKANGTONG MEDICINE TECHNOLOGY CO., LTD, Beijing (CN)

(72) Inventors: Yinglu GUO, Beijing (CN); Zhichao ZHANG, Beijing (CN); Yiming YUAN, Beijing (CN); Nianzeng XING, Beijing (CN); Long TIAN, Beijing (CN); Ke WANG, Beijing (CN); Shengqiang YU, Beijing (CN); Hongjiang ZHAO, Beijing (CN); Xuejun JING, Beijing (CN)

(73) Assignee: BEIJING UNIKANGTONG MEDICINE TECHNOLOGY CO., LTD, Beijing (CN)

(21) Appl. No.: 15/531,930

(22) PCT Filed: Jan. 26, 2016
(86) PCT No.: PCT/CN2016/072130

§ 371 Date: May 31, 2017

(30) Foreign Application Priority Data
Sep. 21, 2015 (CN) 201510602895.0

(51) Int. Cl. A61M 29/02; A61B 17/00

(52) U.S. Cl. CPC A61M 29/02; A61B 17/00234; A61M 2029/025; A61B 2017/00274

(57) ABSTRACT

The invention provides a novel prostate dilation catheter, which comprises a main tube, wherein a urinary catheterization cavity, an internal capsule cavity, an external capsule cavity and a flushing cavity are provided inside the main tube, a composite bar-like high pressure water capsule is arranged in front portions of an outlet of the urinary catheterization cavity and an outlet of the flushing cavity, the composite bar-like high pressure water capsule comprises an internal capsule and an external capsule, the internal capsule is arranged inside the outer capsule and at a front end of the outer capsule, and the front ends of the internal capsule cavity and the external capsule cavity are formed with openings which are located inside the internal capsule and the external capsule respectively. Utilization of such structure makes the catheter will automatically move towards the inside of bladder after a bladder neck is dilated, so as to remove compression on an external sphincter to prevent occurrence of urinary incontinence; and in addition, it is more conducive to preoperative localization and postoperative fixation of the catheter, and can avoid bladder spasm caused by postoperative compression of the catheter on crypt.

### FIELD OF THE INVENTION

The present invention relates to a novel prostate dilation catheter, and particularly to a prostate dilation catheter having a composite bar-like water capsule with an internal capsule located at a front end of an outer capsule.

### BACKGROUND

Benign prostatic hyperplasia (BPH) is a benign lesion common to the aged men. The morbidity thereof is 65% among the people over the age of 60, and 25% of the patients show clinical symptom and require medicine or surgical treatment. Effects of the conventional transvesical prostatectomy or electrotomy and laser surgery are definite, but the patients are dramatically injured by the surgery, and physical tolerance capacity and psychological tolerance capacity of the aged to the surgery are relatively poor, therefore a lot of the aged patients are in fear of surgery. Long term utilization of urinary catheter for maintenance and even only the performance of suprapubic cystostomy to resolve dysuria render the quality of life extremely poor. Using posterior urethra dilation operation for treatment of benign prostatic hyperplasia is a method that should be attached significance to and researched currently in the medical field for treating the benign prostatic hyperplasia.

Prostate dilation catheters in the prior art broadly fall into two types as follows:

1. In order to facilitate insertion, most of prostate dilation catheters in the prior art have an external capsule in a circular shape, spherical shape, arc shape or streamline shape etc., and its structure is as shown in FIG. 1.

2. A prostate dilation catheter of the application with the application No. CN201010578449.8 filed by the present applicant comprises an internal capsule and an external capsule, wherein the internal capsule is located at a front segment inside the external capsule, front ends of the internal capsule cavity and the external capsule cavity are formed with openings which are located inside the internal capsule and the external capsule respectively, the shape of the external capsule after being expanded is bar-like, and its structure is as shown in FIG. 2.

As for the first type of prostate dilation catheter, since the external capsule is located at the bladder neck, when the interior thereof is pressurized, it is not easy to control internal slipping, and the expansion effect is not stable.

As for the second type of prostate dilation catheter, since the external capsule is in a column shape after the expansion, which neither facilitates slipping towards the interior of the bladder during the operation to avoid long-time stress to the external sphincter, nor facilitates postoperative pulling and compression for hemostasis; besides, the internal capsule is located at the rear end of the external capsule, which is unfavorable for preoperative localization and postoperative fixation of the catheter, and there may be a risk of bladder spasm caused by postoperative compression of the internal capsule on crypt.

Regarding the above defects existing in the prior art, the present invention provides a novel prostate dilation catheter, wherein an external capsule of a composite high pressure water capsule is bar-like with a relatively big head end and a relatively small distal end. Utilization of such shape makes the catheter automatically move towards the inside of a bladder after a bladder neck is expanded, so as to remove compression on the external sphincter to prevent occurrence of urinary incontinence; in addition, an internal capsule is located at the head end inside the external capsule, which is more conducive to preoperative localization and postoperative fixation of the catheter, and also avoids occurrence of bladder spasm caused by postoperative compression of the catheter on crypt; meanwhile, since a locating ring is provided at a tail end of the external capsule, whether the catheter moves towards the inside of the bladder can be determined by touching the locating ring.

Through clinical trials, curative effects and security evaluation results of applying the prostate dilation catheter of the present invention to treatment of benign prostatic hyperplasia are as follows: after pulling out the catheter 5-7 days later, all of 35 patients had good micturition, 1 patient had transient urinary incontinence and recovered one month later. The international prostate symptom score was reduced from the preoperative (26.7+2.5) score to (6.1+2.0) score; according to measurement, a residual urine volume measured by B-scan ultrasonography was reduced from the preoperative (132.2+5.2) ml to the postoperative (24.1+3.6) ml; a maximum urine flow rate was increased from the preoperative (6.5+2.3) ml/s to (21.8+2.7) ml/s; all of the comparative differences of various micturition indexes before and after the treatment had statistical significance (p<0.05). Conclusion: the present method is a simple, safe, and effective method, applicable to patients having various degrees of benign prostatic hyperplasia, including middle lobe hyperplasia.

### SUMMARY OF THE INVENTION

An object of the present invention is to provide a novel prostate dilation catheter. The utilization of such shape enables the catheter automatically move towards the inside of a bladder after a bladder neck is expanded, so as to remove compression on the external sphincter to prevent occurrence of urinary incontinence; in addition, it is more conducive to preoperative localization and postoperative fixation of the catheter, and postoperative stimulation of the catheter to crypt can be avoided; meanwhile, since a locating ring is provided at a tail end of an external capsule, whether the catheter moves towards the inside of the bladder can be determined by touching the locating ring.

In order to achieve the above object, technical solutions of the present invention are realized as follows:

A novel prostate dilation catheter, comprising a main tube, wherein a urinary catheterization cavity, a flushing cavity, an internal capsule cavity and an external capsule cavity are provided inside the main tube, a composite bar-like high pressure water capsule is arranged at the back of an outlet of the urinary catheterization cavity and an outlet of the flushing cavity, the composite bar-like high pressure water capsule comprises an internal capsule and an external capsule, the internal capsule is located inside the outer capsule and at a front end of the outer capsule, and the front ends of the internal capsule cavity and the external capsule cavity are formed with openings which are located inside the internal capsule and the external capsule respectively.

Further, the internal capsule injects water into and pressurizes the internal capsule cavity through an internal capsule pressurizing mouth, and the external capsule injects water into and pressurizes the external capsule cavity through an external capsule pressurizing mouth.

Further, the internal capsule is in a spherical shape after pressurization and used for performing location, and the external capsule after pressurization is bar-like with a thick front end and a thin rear end, and used for expanding a bladder neck and an external sphincter.

Further, the inlet of the flushing cavity and the outlet of the flushing cavity are communicated through one flushing tube, and the inlet and the outlet of the urinary catheterization cavity are directly communicated through the whole main tube.

Further, a locating ring or a locating protrusion is provided on the main tube at the back of the external capsule, and it can be revealed under X-ray, and also can be touched when rectal tactus is performed.

Further, the locating ring or the locating protrusion is made of metal material.

Further, the external capsule has a length of 7-13 cm, and the internal capsule has a length of 2-5 cm.

Further, the main tube has a diameter of 5-6 cm, and is made of PVC or PE, and both the internal capsule and the external capsule are made of a composite material.

Further, the internal capsule has a same diameter as a front segment of the external capsule does, and a diameter of a middle segment of the external capsule is 2.5 cm-4 cm.

Further, in operation, a water filling volume of the internal capsule is 20-50 milliliters, and a water filling pressure of the external capsule is 0.3 MPa, and after the operation, a part of water in the external capsule is discharged to lower the pressure of the external capsule to 0.1 MPa for hemostasis.

Compared with the dilation catheters currently available in the market, the present invention has the following advantages: 1. Location of the catheter is precise and convenient. 2. The catheter can automatically move inwards after the prostate is expanded, so as to remove the compression on the external sphincter, and the external sphincter will not be excessively injured, preventing urinary incontinence.

### BRIEF DESCRIPTION OF DRAWINGS

In order to more clearly illustrate technical solutions of embodiments of the present invention or the prior art, figures which are needed for description of the embodiments or the prior art will be introduced briefly below. Obviously, the figures in the following description merely show some embodiments of the present invention, and a person ordinarily skilled in the art can still obtain other relevant figures according to these figures, without paying inventive effort.

Please refer to FIG. 1 to FIG. 6, wherein

FIG. 1 and FIG. 2 are views of the prior art of the present invention;

FIG. 3 is a front view of Example 1 of the present invention;

FIG. 4 is a front view of Example 2 of the present invention;

FIG. 5 is a sectional view of FIG. 3;

FIG. 6 is a sectional view of a main tube.

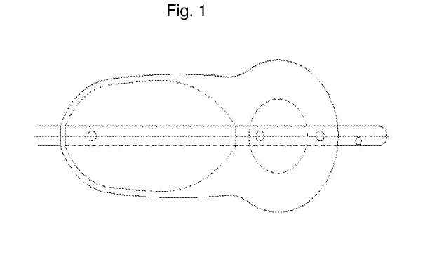

Correspondence of part names and reference signs in FIG. 1 to FIG. 6 is as follows:

1. inlet of urinary catheterization cavity, 2. outlet of flushing cavity,

3. internal capsule, 4. external capsule,

5. internal capsule pressurizing mouth, 6. external capsule pressurizing mouth,

7. inlet of flushing cavity, 8. outlet of urinary catheterization cavity,

9. main tube, 10. locating ring,

11. urinary catheterization cavity, 12. internal capsule cavity,

13. flushing cavity, 14. external capsule cavity,

2a. flushing tube, 3a. internal capsule tube,

4a. external capsule tube.

### DETAILED DESCRIPTION OF THE INVENTION

The technical solutions of the present invention will be described clearly and completely in conjunction with figures below. Apparently, some but not all of examples of the present invention are described. Based on the examples of the present invention, all the other examples, which a person ordinarily skilled in the art obtains without paying inventive effort, fall within the scope of protection of the present invention.

In the description of the present invention, it should be noted that orientational or positional relationships indicated by terms such as “center”, “upper”, “lower”, “left”, “right”, “vertical”, “horizontal”, “inner”, and “outer” and so on are based on orientational or positional relationships as shown in the figures, merely for facilitating describing the present invention and simplifying the description, rather than indicating or suggesting that related devices or elements have to be in the specific orientation or configured and operated in specific orientation, therefore, they should not be construed as limiting the present invention. Besides, terms “first”, “second”, “third” are merely for descriptive purpose, but should not be understood as indicating or suggesting a relative importance.

In the description of the present invention, it should be indicated that unless otherwise specified and defined, terms “installation”, “link” and “connection” should be understood widely, for example, it may be a fixed connection, a detachable connection, or an integrated connection; it may be a mechanical connection, or an electrical connection; it may be a direct connection, or an indirect connection through an intermediate medium; and it also may be an inner communication between two elements. For a person ordinarily skilled in the art, the specific meanings of the above mentioned terms in the present invention can be understood according to specific circumstances.

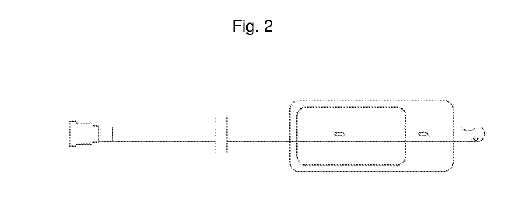

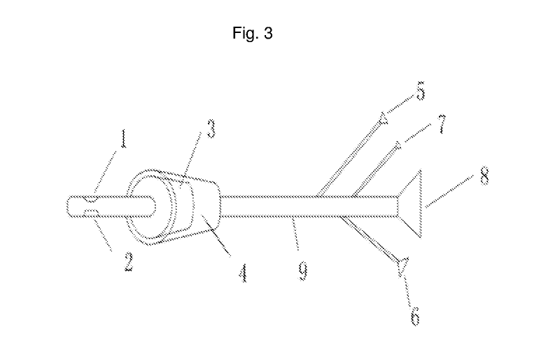

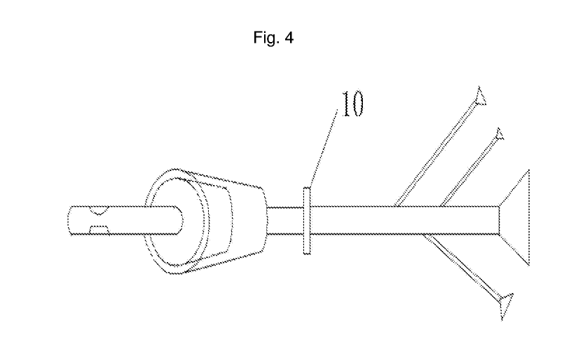

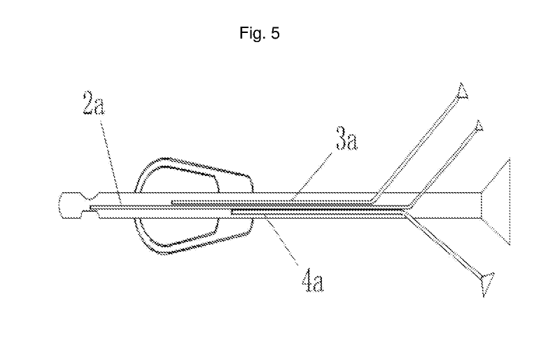

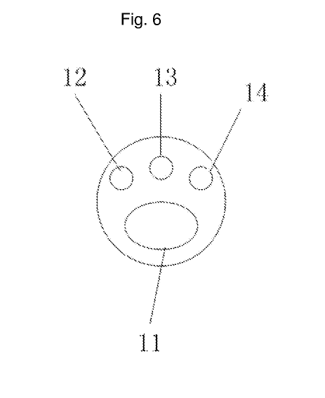

Please refer to FIG. 1 to FIG. 6, wherein FIG. 1 and FIG. 2 are views of the prior art of the present invention; FIG. 3 is a front view of example 1 of the present invention; FIG. 4 is a front view of Example 2 of the present invention; FIG. 5 is a sectional view of FIG. 3, and FIG. 6 is a sectional view of a main tube.

The present invention is further described in detail in conjunction with the figures:

FIG. 1 shows a type of prostate dilation catheter commonly existing in the prior art. In order to facilitate insertion, its external capsule is in a circular shape, spherical shape, arc shape or streamline shape. The catheter in such a shape has the disadvantage that when the interior of the external capsule is pressurized, it is not easy to control internal slipping, and the expanding effect is not stable either.

FIG. 2 shows a prostate dilation catheter of the application with the application No. CN201010578449.8 filed by the present applicant, comprising an internal capsule and an external capsule, wherein the internal capsule is located at a rear segment inside the external capsule, front ends of the internal capsule cavity and the external capsule cavity are formed with openings which are located inside the internal capsule and the external capsule respectively, and the shape of the external capsule after expansion is bar-like. The catheter in such a shape has the disadvantage that the internal capsule is located at a rear end of the external capsule, which is unfavorable for preoperative localization and postoperative fixation of the catheter, and there may be a risk of bladder spasm caused by compression of the internal capsule on crypt.

FIG. 3 is a structural schematic view of a first embodiment of the present invention.

As shown in FIG. 3, a novel prostate dilation catheter comprises a main tube 9, wherein a urinary catheterization cavity 11, a flushing cavity 13, an internal capsule cavity 12 and an external capsule cavity 14 are provided inside the main tube 9, a composite bar-like high pressure water capsule is arranged in back portions of an outlet 8 of the urinary catheterization cavity and an outlet 7 of the flushing cavity, the composite bar-like high pressure water capsule comprises an internal capsule 3 and an external capsule 4, the internal capsule 3 is located inside the outer capsule 4 and at a front end of the outer capsule 4, and the front ends of the internal capsule cavity 12 and the external capsule cavity 14 are formed with openings which are located inside the internal capsule 3 and the external capsule 4 respectively.

FIG. 4 is a structural schematic view of a second embodiment of the present invention.

As shown in FIG. 4, on the basis of the first embodiment, further, a locating ring or a locating protrusion 10 is provided on the main tube 9 at the back of the external capsule 4, and it can be revealed under X-ray and also can be touched when rectal tactus is performed. Further, the locating ring or the locating protrusion 10 is made of metal material.

FIG. 5 and FIG. 6 are views revealing the internal structure of the main tube of the present invention.

As shown in FIG. 5 and FIG. 6, the urinary catheterization cavity 11, the flushing cavity 13, the internal capsule cavity 12 and the external capsule cavity 14 are provided inside the main tube 9, the flushing cavity 13 communicates the inlet 7 of the flushing cavity and the outlet 2 of the flushing cavity through one flushing tube 2a, and the inlet 1 of the urinary catheterization cavity and the outlet 8 of the urinary catheterization cavity are directly communicated through the whole main tube 9. The internal capsule 3 injects water into and pressurizes the internal capsule tube 3a through the internal capsule pressurizing mouth 5, and the external capsule 4 injects water into and pressurizes the external capsule tube 4a through the external capsule pressurizing mouth 6.

The internal capsule 3 is in a spherical shape after pressurization and used for performing location, and the external capsule 4 after pressurization is bar-like with a thick front end and a thin rear end, and used for expanding the bladder neck and the external sphincter.

The external capsule has a length of 7-13 cm, and the internal capsule has a length of 2-5 cm. The internal capsule has the same diameter as the front end of the external capsule does, and the diameter of a middle segment of the external capsule is 2.5 cm-4 cm.

The main tube has a circumference of 5-6 cm, and is made of PVC or PE. Both the internal capsule and the external capsule are made of a composite material.

In practical surgery, a specific operation of the present dilation catheter is as follows:

The catheter of the present invention is designed with two capsules, namely, the internal capsule and the external capsule, and one locating ring, the internal capsule is in a spherical shape and used for performing location, and the external capsule is bar-like and used for expanding the bladder neck and the external sphincter.

In operation, the catheter is transurethrally inserted into the bladder, firstly, 20 milliliters of physical saline is injected into the internal capsule, the internal capsule is in a spherical shape after being filled with water, and then, the catheter is pulled outwards to locate. After location, physical saline is filled into the external capsule through the external capsule pressurizing mouth by a pressurized apparatus which can control the pressure until the surface pressure of the external capsule reaches 0.3 MPa, with the expansion of the external capsule, the bladder neck and the external sphincter are expanded, meanwhile the prostate and the surgical coating are expanded, subsequently, a part of water in the external capsule is discharged to lower the pressure of the external capsule to 0.1 MPa for hemostasis.

Since the external capsule after being filled with water is bar-like with a relatively thick front end and a relatively thin rear end, the catheter will automatically move towards the inside of the bladder after the bladder neck is expanded, so as to remove compression on the external sphincter to prevent the occurrence of urinary incontinence. Meanwhile, whether the catheter moves towards the inside of the bladder can be determined by touching the locating ring.

Six hours after the surgery, water in the external capsule can be completely discharged and the pressure of the internal capsule is maintained for fixing the catheter. Since the internal capsule is located at the head end inside the external capsule, it is more conducive to preoperative localization and postoperative fixation of the catheter, and also avoids the occurrence of bladder spasm caused by postoperative compression of catheter on crypt.

The main pathological feature of hyperplasia is interstitial hyperplasia formed by fibrillary muscle. Hyperplastic glands protrude towards both sides and the inside of the bladder, so that the posterior urethra prolongs and becomes narrow, and resistance at an outlet of the bladder is increased. Based on the above pathogenesis, with the dilation catheter of the present invention, the high pressure water capsule after being injected with the physical saline can bluntly tear the prostate surgical coating from the front, glandular leaves at both sides are also subsequently opened, and a surgeon inserts an index finger into the rectum and can visually feel morphologic changes of the front portion of the prostate before and after the dilation. Thereafter, the torn prostate tissues are subject to inflammatory infiltration, the denaturation of smooth muscle fibers and the reduction of collagen content make them unable to retract. The surrounding tissues are embedded into the torn prostate coating, and the torn glands cannot be closed, so that one cavity gap wide at the top and narrow at the bottom is formed at an internal urethra orifice, and long term unblocking of the urethra is realized.

Compared with the dilation catheters currently available in the market, the present invention has the following advantages:

1. The external capsule after being filled with water is bar-like with a relatively thick front end and a relatively thin rear end, therefore, the catheter will automatically move towards the inside of the bladder after the bladder neck is expanded, so as to remove compression on the external sphincter to prevent the occurrence of urinary incontinence.

2. The internal capsule is located at the front end inside the external capsule, which is conducive to preoperative localization and postoperative fixation of the catheter, and effectively avoids bladder spasm caused by postoperative compression on crypt

3. The locating ring or the locating protrusion is provided on the main tube at the back of the external capsule, and it can be revealed under X-ray and also can be touched when rectal tactus is performed, and whether the catheter moves towards the inside of the bladder can be judged by touching the locating ring.

Judging by the surgical treatment and follow-up results of 35 BPH patients in the present group: transurethral treatment to the BPH patients with the bar-like water capsule prostate dilation operation has significant short and mid-term curative effects, and this surgical method is highly safe, relatively low-cost, worth clinical popularization and application, and applicable to different grades of hospitals.

Finally, it is to be explained that the above various examples are merely to illustrate the technical solutions of the present invention, rather than limiting the invention; while detailed description is made to the present invention with reference to the above-mentioned examples, those ordinarily skilled in the art should understand that the technical solutions of the above-mentioned examples can be modified, or equivalent substitutions can be made to some or all technical features therein; and with these modifications or substitutions, the essence of the corresponding technical solutions does not depart from the scope of the technical solutions of the examples of the present invention.

1. A prostate dilation catheter comprising:
a main tube, in which a urinary catheterization cavity a flushing cavity, an internal capsule cavity and an external capsule cavity are provided;
a composite bar-like high pressure water capsule being arranged at the back of an outlet of the urinary catheterization cavity; and
an outlet of the flushing cavity,
wherein,
the composite bar-like high pressure water capsule comprising an internal capsule and an external capsule;
the internal capsule is located inside the outer capsule and at a front end of the outer capsule; and
the front ends of the internal capsule cavity and the external capsule cavity are formed with openings which are located inside the internal capsule and the external capsule respectively.

2. The prostate dilation catheter according to claim 1, wherein the internal capsule injects water into and pressurizes the internal capsule cavity through an internal capsule pressurizing mouth, and the external capsule injects water into and pressurizes the external capsule cavity through an external capsule pressurizing mouth.

3. The prostate dilation catheter according to claim 2, wherein the internal capsule is in a spherical shape after pressurization and used for performing location, and the external capsule after pressurization is bar-like with a thick front end and a thin rear end, and used for expanding a bladder neck and an external sphincter.

4. The prostate dilation catheter according to claim 1, wherein in the flushing cavity, an inlet of the flushing cavity and the outlet of the flushing cavity are communicated through one flushing tube, and the inlet and the outlet of the urinary catheterization cavity are directly communicated through the whole main tube.

5. The prostate dilation catheter according to claim 1, wherein a locating ring or a locating protrusion is provided on the main tube, at back of the external capsule, and is capable of being touched when rectal tactus is performed.

6. The prostate dilation catheter according to claim 5, wherein the locating ring or the locating protrusion is made of metal material, and is capable of being revealed under X-ray.

7. The prostate dilation catheter according to claim 1, wherein the external capsule has a length of 7-13 cm, and the internal capsule has a length of 2-5 cm.

8. The prostate dilation catheter according to claim 1, wherein the main tube has a diameter of 5-6 cm, and is made of PVC or PE, and both the internal capsule and the external capsule are made of a composite material.

9. The prostate dilation catheter according to claim 8, wherein the internal capsule has a same diameter as a front segment of the external capsule, and a diameter of a middle segment of the external capsule is 2.5 cm-4 cm.

10. The prostate dilation catheter according to claim 2, wherein a water filling volume of the internal capsule is 20-50 milliliters, and a water filling pressure of the external capsule is 0.3 MPa.
